(19) United States
(12) Patent Application Publication
(10) Pub. No.: US 2026/0283575 A1
(43) Pub. Date: Sep. 24, 2026

(54) DETERMINATION OF VESSEL GEOMETRY AND FLOW PARAMETER

(71) Applicant: Siemens Healthineers AG, Forchheim (DE)

(72) Inventors: Dominik Neumann, Erlangen (DE); Alexandru Turcea, Busteni (RO); Serkan Cimen, West Orange, NJ (US); Martin Berger, Bubenreuth (DE)

(21) Appl. No.: 19/572,823

(22) Filed: Mar. 19, 2026

(30) Foreign Application Priority Data
Mar. 20, 2025 (EP) 25465518

(51) Int. Cl. A61B 6/50; A61B 6/00; G06T 7/00; G06T 7/62; G06T 7/64

(52) U.S. Cl. CPC A61B 6/504; A61B 6/4441; A61B 6/5217; A61B 6/5235; G06T 7/0012; G06T 7/62; G06T 7/64; G06T 2207/10116; G06T 2207/20; G06T 2207/30096; G06T 2207/30104

(57) ABSTRACT

A method includes receiving a plurality of images of vessels obtained from different viewing angles relative to the vessels. In each image and for a first of the vessels, a distance of different points along the first vessel relative to a reference location at the first vessel is determined, and in each image, and at each of the different points, a parameter of the first vessel for blood flow through the first vessel is determined. This provides, for each image, a data sequence indicating the parameter relative to a distance to the reference location. A sequence alignment of the data sequences obtained for the plurality of images is carried out, providing aligned data sequences. An aggregated representation of the first vessel is determined based on the aligned data sequences, and a flow parameter is determined for the first vessel from the aggregated representation.

This application claims the benefit of European Patent Application No. EP 25465518, filed on Mar. 20, 2025, which is hereby incorporated by reference in its entirety.

### TECHNICAL FIELD

Various examples of the disclosure generally relate to images showing vessels (e.g., obtained during angiography). Various examples specifically relate to determining an aggregated representation of a vessel and to determining a flow parameter from the aggregated representation.

### BACKGROUND

Coronary X-ray angiography may be used for the assessment of coronary arteries and the diagnosis of coronary artery disease (CAD). For the functional assessment of coronary artery stenosis, the Fractional Flow Reserve (FFR) index may be used. FFR is a hemodynamic index to determine the hemodynamic significance of a given epicardial coronary stenosis. It may be defined as the ratio of maximal achievable blood flow in a myocardial bed in the presence of an epicardial stenosis to the theoretical normal maximal flow in the same myocardial distribution.

Angio-based coronary assessment pipelines may employ a 3D reconstruction step with a goal to reconstruct the 3D vessel geometry. However, a full 3D image dataset showing the organ under examination with sufficient resolution in all three dimensions is not always available.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an improved method of determining flow parameters of vessels is provided.

According to a first aspect, a computer-implemented method is provided. The computer-implemented method includes receiving a plurality of images of vessels, where the plurality of images were obtained from different viewing angles relative to the vessels. The act of receiving a plurality of images does not contain the act of obtaining these images (e.g., by a medical imaging device); however, receiving a plurality of images may be understood as receiving image data of such images that have been previously obtained with an imaging device. In each image of the plurality of images, a distance of different points of a first vessel from the vessels is determined along the first vessel relative to a reference location at the first vessel. Further, in each of the images and at each of the different points, a parameter of the first vessel for the blood flow through the first vessel is determined, so that for each of the images, a data sequence is provided describing the parameter relative to the distance to the reference location. The reference location, which may be a reference point at the vessel, may be a tip of a catheter or any anatomic location such as a bifurcation of the vessel. In addition, a sequence alignment of the data sequence obtained for the plurality of images is carried out providing aligned data sequences. Based on the aligned data sequences, it is possible to determine an aggregated representation of the first vessel, and a flow parameter of the first vessel may be determined from the aggregated representation. Thus, the present embodiments may provide a simplified technique for determining a vessel representation and a flow parameter (e.g., a hemodynamic parameter) within the vessel (e.g., when no full 3D dataset of the vessel or the organ under examination is available).

In this application, vessels are blood vessels. Blood vessels are the tubular structures of a circulatory system that transport blood throughout a vertebrate's body. There are five types of blood vessels: the arteries that carry the blood away from the heart; the arterioles; the capillaries, where the exchange of water and chemicals between the blood and the tissues occurs; the venules; and the veins that carry blood from the capillaries back towards the heart.

The viewing angle relative to the vessels is the angle from which the image of the vessels has been taken. This may depend on the rotatory angle of a source and/or a detector of a medical imaging device. In such a medical imaging device, the source and/or detector may be mounted in a rotatable manner, making the source and/or detector rotatable by certain angles, the viewing angles, around an object to be imaged, such as a human or animal body.

In act 320, for the detected vessel, the distance of each point along the vessel relative to a reference point, referred to as reference location, is determined. The reference location may be a tip of the catheter or any other anatomic location, such as a bifurcation that may be determined easily and without doubt in the images. For example, the reference location is any location in the image that may be detected in the images with a high reproducibility and likelihood.

The data sequence may describe the radius evolution for different distances to the reference location. The data sequence corresponds to a sequence of data points that describe the parameter, such as the available cross cross-sectional area available for the blood at the corresponding location relative to the reference location.

Further, a computing device including a processing unit and a memory with program code is provided. The processing unit, when executing the program code, is configured to perform a method as discussed above or as discussed in further detail below. Additionally, an angiography device including the computing device is provided. With the above-described method and computing device, it is possible to determine the flow parameter in the first vessel without the need to generate a full 3D model of the area surrounding the first vessel. Based on the flow parameter, it may be determined whether further examination of the corresponding vessel is necessary or not.

Further, it is possible to also determine in each image of the plurality of images, for the first vessel, at least one additional feature of the first vessel and its distance to the reference location. The sequence alignment is carried out taking into account the distance of the at least one additional feature to the reference location. The distance of the feature to the reference location is the distance of the location of the vessel at which the feature is determined. This additional feature may help to further improve the alignment of the plurality of images relative to each other. When the alignment is improved, the accuracy of the aggregated representation is improved, and thus the determination of the flow parameter.

The additional feature may include features of the first vessel such as a bifurcation occurring at the first vessel, a vessel segment label, or a stenosis marker or the like. These features such as a bifurcation or stenosis marker may be either detected with a high precision and high reproducibility or may help to correctly identity a vessel in case of a vessel segment label. As indicated above, the improved aggregated representation helps to precisely determine the flow parameter.

The parameter may represent a cross-sectional area available for the blood flow through the first vessel. The parameter may be a parameter representing a surface or a cross-sectional area available for the blood flow in the first vessel. The cross-sectional area is a valid parameter describing the hydraulic conditions present in the vessel. The parameter may include a diameter or radius of the first vessel. When the cross-sectional area, the diameter or the radius of the first vessel is known, a quantification of the blood flow through the first vessel becomes possible, and flow parameters such as fractional flow reserve may be determined.

The act of determining the aggregated representation may include the act of aggregating features of the first vessel belonging to the same location. Aggregating features of the first vessel belonging to the same location includes aggregating the features over at least two images taken from different viewing angles. The act of determining the aggregated representation may include the act of aggregating locations of the first vessel belonging to the same location. When aggregating the same locations, geometric information of the first vessel is aggregated so that a picture of geometry of the first vessel is obtained. The geometry of the first vessel helps to understand and correctly interpret the flow information present in the corresponding vessel.

It is possible to aggregate the parameter as geometric mean. For the cross-sectional area, for example, the geometric mean is a valuable parameter that helps to correctly represent the geometry present in the vessel. It may help to remove outliers determined in the parameter and helps to more precisely describe the geometry available for the flow.

Determining the aggregated representation may include the act of aggregating distances belonging to the same location, where the distances to the reference location are aggregated with aggregating a maximum difference between a current location and a previously aggregated location. When the maximum difference is used and aggregated, the result may be a more accurate estimate of the real 3D distance, as a 3D segment length determined in a 2D projection of an image is less or equal to the real 3D length.

It is further possible to determine for each image of the plurality of images, a curvature of the first vessel along the different points. Determining the aggregated representation includes aggregating the curvature at each of the different points. The curvature is a parameter that helps to precisely determine a representation of the vessel and especially the cross section available for the flow. As a consequence, the flow parameter may be assessed more precisely.

Determining the flow parameter may include determining a fractional flow reserve (FFR), an index of microcirculatory resistance (IMR), microvascular resistance reserve (MRR), or any combination thereof. All these parameters are important parameters in a diagnosis of a vessel disease.

In one embodiment, the images are 2D X-ray images (e.g., X-ray projection images). In one embodiment, the images are fluoroscopy images. The images may include angiographic X-ray images of the heart. Coronary X-ray is the method of choice for the assessment of coronary arteries and the diagnosis of coronary artery disease, and the method described above or in further detail below may be applied to those images.

For the sequence alignment, it is possible to use methods such as Dynamic Time Warping and Optimal Subsequence Bijection. Both methods may provide good results in the present field of application.

Further, a computer program including program code to be executed by a processing unit that, when executed by the processing unit causes the processing unit to carry out a method as discussed above or as discussed in further detail below, is provided.

Finally, a carrier including the computer program, where the carrier is an electronic signal, an optical signal, a radio signal, a computer-readable storage medium (e.g., a non-transitory computer-readable storage medium) is provided.

With the method discussed above, it is possible to determine a flow parameter or hemodynamic parameter for a vessel without the need to have a full three-dimensional data set and/or without the need of a traditional 3D reconstruction (e.g., based on multiple 2D views). This becomes possible by aggregating information of the vessel from multiple views. It has been observed that a full three-dimensional model of the vessel is not required for most of the applications. With the determination of the parameter at the different points relative to a reference location and the sequence alignment, an aggregated representation of the vessel that is accurate enough to precisely determine a flow parameter such as a fractional flow reserve (FFR) or any other flow parameter for a vessel is obtained.

With the method as per the present embodiments, a flow parameter may be determined based on two-dimensional fluoroscopy images without the need to create based on 2D images of the object a three-dimensional data set of the object with almost isotropic resolution in all directions. In one embodiment, this step is omitted, using the aggregation and the sequence alignment as described.

It is to be understood that the features mentioned above and features yet to be explained below may be used not only in the respective combinations indicated, but also in other combinations or in isolation without departing from the scope of the present application. Features of the above-mentioned aspects and embodiments described below may be combined with each other in other embodiments unless explicitly mentioned otherwise.

### BRIEF DESCRITION OF THE DRAWINGS

The foregoing and additional features and effects of the application will become apparent from the following detailed description when read in conjunction with the accompanying drawings in which like reference numerals refer to like elements.

FIG. 1 shows a schematic view of an angiography device configured to generate images of vessels for which a flow parameter may be determined.

FIG. 2 shows two angiographic images of the same person acquired with different acquisition geometry.

FIG. 3 shows a schematic view of one of the vessels being highlighted but viewed from different angulations.

FIG. 4 shows a schematic view of a geodesic distance in the vessel relative to a reference location.

FIG. 5 shows a schematic view of the extracted lumen contours used to determine a radius for the different views together with the determined geodesic distance.

FIG. 6 shows a schematic processing flow how a flow parameter may be determined for the vessel.

FIG. 7 shows a schematic view of a sequence alignment used to determine aggregated information for the vessel.

FIG. 8 shows a schematic view of a flowchart including acts used to determine a flow parameter for a vessel.

### DETAILED DESCRIPTION

In the following, embodiments will be described in detail with reference to the accompanying drawings. It is to be understood that the following description of embodiments is not to be taken in a limiting sense. The scope of the invention is not intended to be limited by the embodiments described hereinafter or by the drawings, which are to be illustrative only.

The drawings are to be regarded as being schematic representations, and elements illustrated in the drawings are not necessarily shown to scale. Rather, the various elements are represented such that their function and general purpose becomes apparent to a person skilled in the art. Any connection or coupling between functional blocks, devices, components of physical or functional units shown in the drawings and described hereinafter may also be implemented by an indirect connection or coupling. A coupling between components may be established over a wired or wireless connection. Functional blocks may be implemented in hardware, software, firmware, or a combination thereof.

Independent of the grammatical term usage, individuals with male, female, or other gender identities are included within the term.

In the following a solution is discussed where geometric information of vessels is aggregated from multiple views having different angular settings, while keeping preconditions such as minimum angular separation and full visibility of the vessels in all the used views to a minimum.

The present embodiments will be discussed in connection with angiographic X-ray images obtained by an angiography device; however, any other imaging system may be used where images of an organ with vessels may be obtained and where not a full 3D dataset with similar resolution in all three directions is available, but only 2D images obtained at different angles relative to a region of interest. Further, the images are images of the heart showing the coronary arteries; however, any other organ with vessels may be used such as liver or kidney. In general, the images shown vessels representing tube-like structures that carry fluids, especially blood.

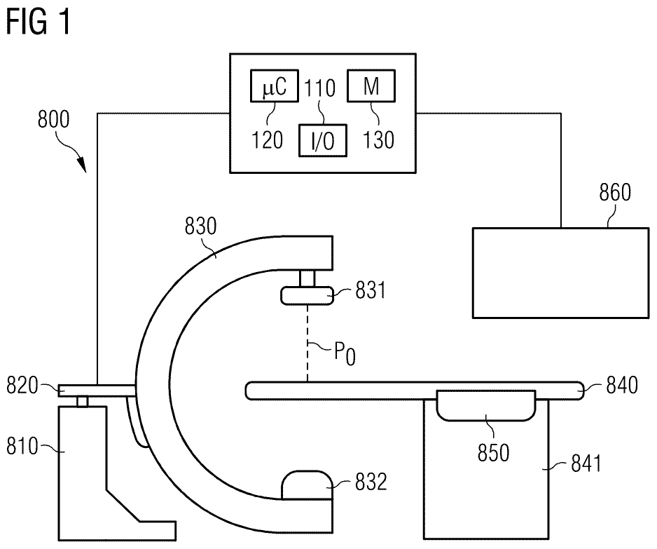

As an example angiography device, FIG. 1 schematically illustrates aspects with respect to a C-arm machine 800. The C-arm machine 800 may be used for both angiography and fluoroscopy. In FIG. 1, the C-arm machine 800 is in neutral position P₀. It is possible to manipulate the C-arm machine 800 to be in a rotated position deviating from the neutral position P₀. The angle between the neutral position P0 and a specific rotated position is referred to as the angiography angle or the fluoroscopy angle. The C-arm machine 800 includes a rotatable C arm 830 on which X-ray emission means 831 (e.g., an X-ray emitter) and X-ray detection means 832 (e.g., an X-ray detector) may be mounted. The C arm 830 and thereby X-ray emission means 831 and X-ray detection means 832 are positioned to center around patient surface 840. X-ray emission means 831 may emit X-rays that penetrate through a patient positioned on the patient surface 840. X-ray detection means 832 detects the X-rays emitted from X-ray emission means 831. When a patient on the patient surface 840 is injected with a radio-opaque contrast agent into the patient's vessels, some of the X-rays emitted by X-ray emission means 831 are absorbed by the radio-opaque contrast agent, leading X-ray detection means 832 to detect a sequence of images of the vessels filled with the radio-opaque contrast agent (e.g., an angiogram). X-ray emission means 831 and X-ray detection means 832 may also collectively be referred to as X-ray imaging means. As a consequence, with the different position of the C arm 830, images that show the patient's vessels in different viewing angles are obtained.

The C arm 830 may be coupled to a C arm rotation unit 820. The C arm rotation unit 820 may be any motorized means (e.g., a motorized device or motor) configured to rotate the C arm 830 according to an angiography angle or a fluoroscopy angle. The C arm rotation unit 820 may be attached to and controlled by a C arm control until 810. The C arm control unit 810 may be any kind of circuitry capable of controlling C arm 830. For example, the C arm control unit 810 may include a computing device (e.g., a computer having one or more processors).

The C-arm machine 800 (e.g., device) may further include a control panel 850 mounted onto a side surface of patient surface support 841. The control panel 850 may be used to control C arm 830 in order to guide medical specialists to pathological vessels. FIG. 1 does not show any connections between control panel 850 and C arm 830 to simplify the depiction of the example C-arm machine 800. In some examples, the connection may be wireless. In some further examples, the connection may be wired and may, for example, be integrated into the ceiling of the room where the C-arm machine 800 is located.

The device 800 may include a central computing device 100 (e.g., a computing device) configured to control the device 800. The computing device 100 includes an interface 110 that may be provided for transmitting data or control messages to other entities, and for receiving data and control messages from other entities. The computing device 100 also includes a processing unit 120 that is responsible for the operation of the computing device 100. The processing unit 120 includes one or more processors and may carry out instructions stored on a memory 130. The memory 130 may include a read-only memory, a random access memory, a mass storage, a hard disk, or the like. The memory 130 may also include suitable program code to be executed by the processing unit 120 so as to implement the functionalities described in further detail below where flow information for a vessel may be determined based on the images generated by device 800.

The C-arm device 800 may also include a display 860. The display 860 may be used to display information to the medical specialist, such as a real-time X-ray image with an overlaid vessel roadmap image including a path to one or more pathological vessels, and/or a location of the pressure wire for measuring the at least one measurement associated with the at least one hemodynamic index. Further, the display 860 may be used to display vessel segmentation data included in overlaid vessel roadmap images, including labels for various vessel segments. In some examples, the display 860 may be a touch screen, which may be used to toggle the display of the vessel segmentation data on and off.

The C-arm machine 800 may be connectable to a database (not shown in FIG. 1), such as a PACS located within a local network of a hospital, for storing angiograms and/or measurements associated with the at least one hemodynamic index.

According to various examples, a name (e.g., of a specific segment of a coronary artery of interest or of the entire coronary artery of interest) may be determined based on a region of interest of the angiography exam. For example, such a region of interest may include the left or right branch of the coronary vessel tree, or a specific vessel section. The region of interest may be determined based on user input, or on a patient's electronic medical record included in a health information system.

For example, the coronary artery of interest within the region of interest may be one or more segments of the coronary arteries of the heart.

In the following, an alternative to the 3D reconstruction task is discussed, based on the observation that a full 3D model is not required for most of the angio-based coronary assessment tasks. Instead, obtaining accurate cross-sectional diameters and/or cross-sectional areas of the target vessel along with accurate distances between these cross-sections is sufficient. Additional geometric features such as local curvature of the vessel may also be obtained with the method of the present embodiments. The output of the method of the present embodiments is thus a 2.5D geometry that lacks 3D locations such as centerline points and lumen meshes but contains sufficient information for performing the desired coronary assessment.

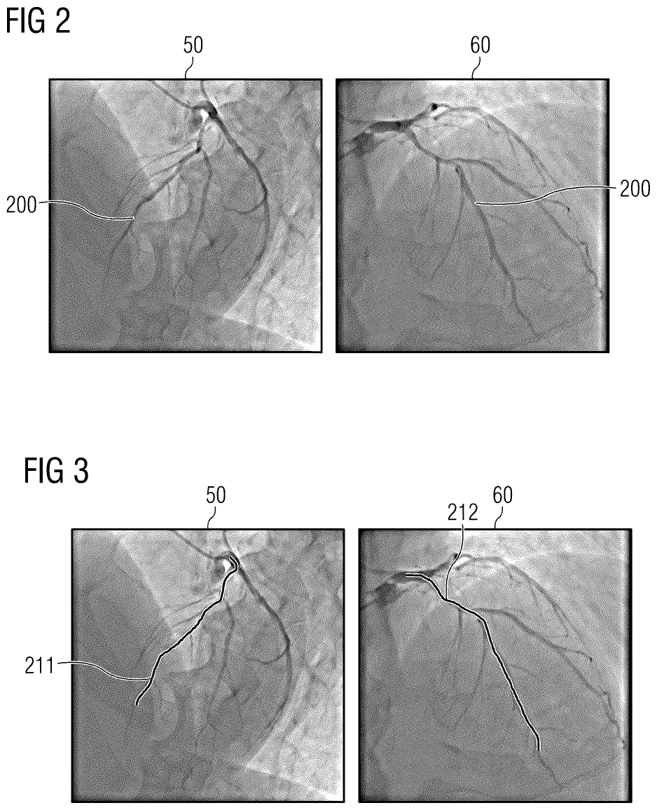

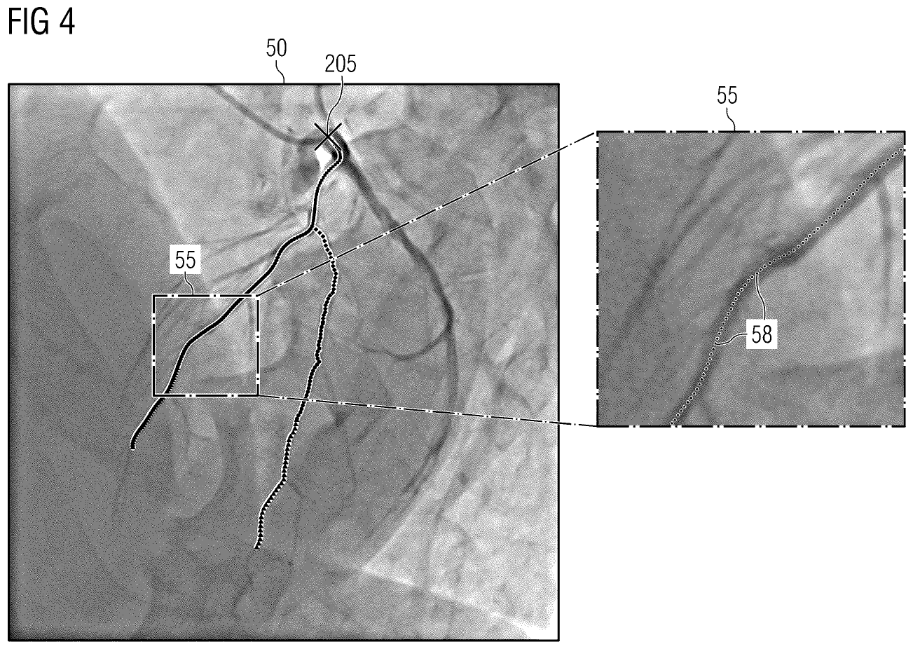

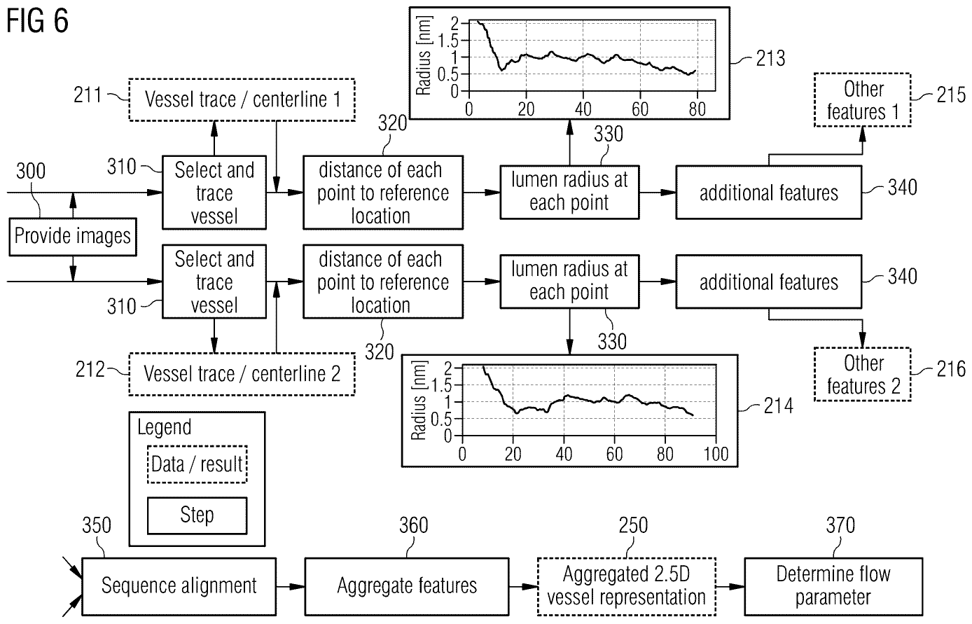

FIG. 2 shows a schematic view of two angiographic images 50 and 60 of the same patient, however, acquired with a different acquisition geometry. Both images show the same vessel 200 that is represented differently in the images 50 and 60 in view of the different imaging angle of the C-arm. In the following, it will be discussed in connection with FIG. 6 in more detail how flow information such as the fractional flow reserve may be determined based on different views of an organ or a body part comprising vessels such as an artery. In addition to FIG. 6, reference will be made to the images shown in FIGS. 2 to 5 for further explanation. The present embodiments are discussed in connection with two different views or sequences (e.g., where each view includes a full angiography sequence including multiple images, also referred to as image frames). The number of 2D images depends on the time duration of the scan (e.g., in the order of 5 to 15 s, and the image frame rate ca be between 7 to 15 frames per second so that each view may include between 50 and 200 (image) frames). Each view is acquired based on a specific acquisition geometry such as a different viewing angle. In the present case, two or more views are used, each including multiple frames. Referring to FIG. 6, this provides that these views or sequences including 50 and 60 are provided in act 300 to the computing device 100 where the processing discussed in FIG. 6 may be carried out. In act 310, in each of the images 50 and 60, a first vessel (e.g., or simply referred to as vessel hereinafter) is selected such as the vessel 200 shown in FIG. 2, where the same vessel is selected in both images. The vessel may be identified based on known image post-processing methods. As a result, a vessel trace or centerline 211 is provided in each of the images (e.g., the traces 211 and 212 as shown in FIG. 3). In act 320, for the detected vessel, the distance of each point along the vessel relative to a reference point referred to as reference location is determined. The reference location may be a tip of the catheter or any other anatomic location such as a bifurcation that may be determined easily and without doubt in the images. Referring to FIG. 4, a reference location 205 is shown, such as the catheter tip, and the path along the vessel is divided into several points that may have a distance of one or several millimeters or even in the sub-millimeter range (e.g., 0.2 to 0.4 mm) depending on the provided solution.

FIG. 4 also shows an enlarged section 55 of the image 50 showing the sequence of points 58, where for all of these points, a distance relative to the reference location 205 is determined. In this act, for example, the projected two-dimensional geodesic distance from the reference location to each point on the vessel is determined. After this act 320, the contours are determined at each of the locations in order to extract a lumen radius in act 330. As a result, a sequence of parameters is obtained such as a diagram that indicates the radius or diameter relative to the reference location. This diagram is shown in FIG. 6 for the two vessel orientations by the sequences 213 and 214.

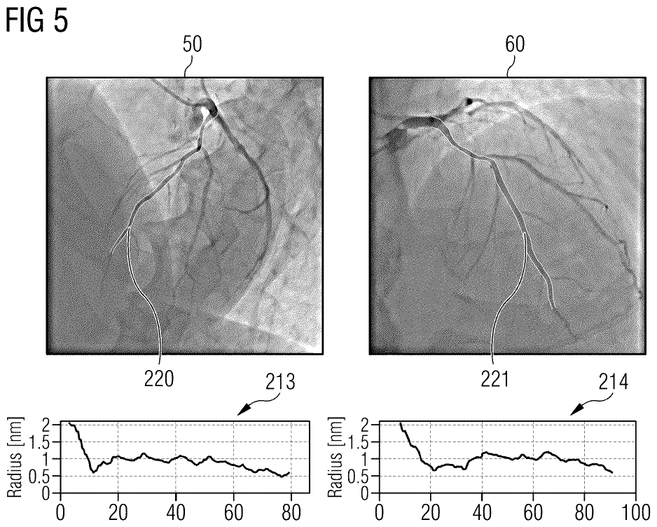

Referring additionally to FIG. 5, the contours 220 and 221 and the lumen radius along the vessel at the different points are determined. The result of the radius calculation is shown in the diagram in the lower part of FIG. 2, the sequence 213 that describes the radius in millimeters relative to the distance to the reference location where the sequence 213 shows the curve for the image 50 and the sequence 214 shows the same situation for another projection but the same vessel. This step includes a vessel segmentation and lumen radius extraction at each of the points along the vessel. Referring again to FIG. 6 after this act 330 where a surface area, a cross-sectional area, or radius of the vessel is determined, it is possible in an optional step to use additional features for each point on the vessel trace such as the local curvature of the vessel, where for the same horizontal axis, x-axis, the distance is used. This act 340 of the detection of additional features along the vessel provides the additional features such as curvature, the features 215 and 216. After act 340, a sequence of data is obtained for each of the images, and in act 350, it is possible to carry out a sequence alignment between the different obtained sequences. The sequence alignment may include an algorithm such as Dynamic Time Warping, DTW, or Optimal Subsequence Bijection or any of its variants. This sequence alignment provides a mapping or correspondence between the locations in the views by computing the optimal alignment cost of the provided sequences. The sequence alignment method leads to aligned sequences for the radius. Here, a mapping may be determined between each point on the first sequence to a point on the second sequence such that some error metric based on the radius and potentially other features is minimized. In act 360, additional features may be used for the alignment such as vessel segment labels, or a stenosis marker or bifurcation locations may be included as inputs to the sequence alignment algorithm. Further, it is possible that each feature is attributed a different importance by scaling its associated error cost when optimizing the mapping function between the different sequences.

Once an optimal mapping function is obtained, features for which it is determined that they pertain to the same location on the vessel of interest are aggregated in order to arrive at an aggregated vessel representation 250 shown in FIG. 6. The mapping of act 350 is then used for the aggregation. Depending on the selected feature, an aggregation may be carried out differently depending on the parameter. By way of example, the diameters may be aggregated either via a geometric mean operation where the resulting value may be considered as an equivalent to the hydraulic diameter at that location, or one may compute the equivalent hydraulic area. The distances to the reference location may be aggregated by adding up the maximum difference between the current mapped locations and the previously aggregated location. This resulting value may be a more accurate estimate of the real 3D distance. This is based on the fact that a 3D segment length measured in a 2D projection is less or equal to the real 3D segment length. Similar aggregation functions may be devised for other features. The result of acts 350 and 360 are aligned data sequences that are aligned relative to one another, such that the same location at the vessel is provided at the same distance relative to the reference location.

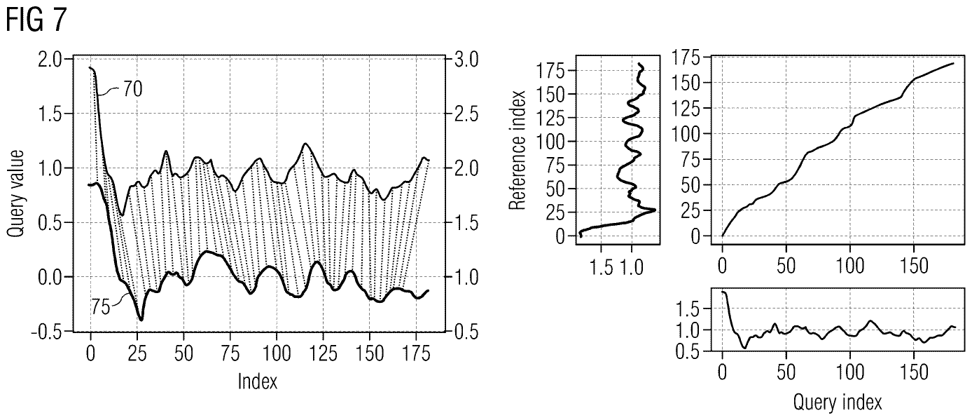

FIG. 7 shows a schematic view how a sequence of values such as the radius for one image may be aligned to the sequence 75 of another image. On the right hand side along the y axis of the upper two plots is the reference sequence (e.g., the radius profile of the first view) and along the x axis the query sequence (e.g., the radius profile from the second view). In the center plot of the right-hand side, the mapping path representing the minimum cost is shown (e.g., the result of DTW for these two sequences). From that one, the mapping index in query-index in reference may be directly read. The result of the alignment is a, for example, 2.5D representation for an aggregated vessel representation 250 for the vessel of interest with cross-sections separated by the estimated 3D distance between them and a hydraulic diameter or areas that may then be directly used as input for the determination of a flow parameter in act 370 such as the fractional flow reserve computation and a quantitative coronary angiography (QCA).

The sequence mapping carried out in acts 350 and 360 may be further improved by augmenting the error function to be optimized with additional terms based on parameters such as reprojection errors. This may be achieved by computing the epipolar line associated to each location in one view onto the other views, computing the intersection of the epipolar line to the target vessel in each view, computing the epipolar line from the intersection points back to the initial view, computing the intersection of the epipolar line to the target vessel in the initial view, and/or computing the distance between the intersection point and the initial location.

With the inclusion of these additional error terms, mapping function optimization may also consider acquisition geometry. A global translation error occurring due to table movement or other factors may be cancelled out by mean-centering the computer distances or using other regularization methods. The method discussed above also supports stitching, which provides that not the complete length of the vessel is provided in one of the images, but only part of it.

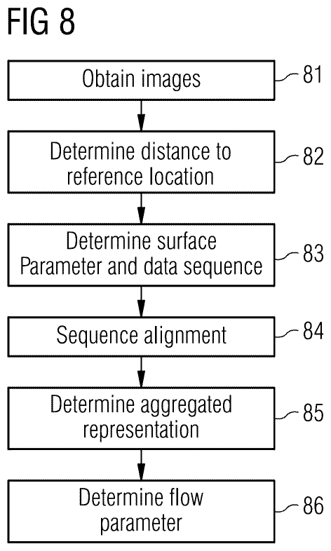

FIG. 8 summarizes some of the acts carried out in the above discussed method. In act 81, at least two images or a plurality of images of a body that indicate vessels (e.g., angiographic images of the heart, where the images were generated with different viewing angles relative to the object under examination) are obtained. Referring to FIG. 1, this provides that the C-arm has different rotation angles. Obtaining the images may include retrieving or selecting the images from a storage medium such as memory 130. The images obtained from the body include vessel representations (e.g., obtained with the angiographic device 800 shown in FIG. 1). In act 82, in each of the images and for one of the vessels, a distance of different points along the first vessel relative to a reference location is determined. The reference location is any location along the vessel that may be precisely determined in the images. This was discussed above in connection with act 320 and FIG. 4. Further, for each of the points, a parameter such as a radius or diameter is determined in act 83, and the result is a data sequence describing the radius evolution for different distances to the reference location. The data sequence corresponds to a sequence of data points that describe a parameter such as the available cross-sectional-area available for the blood at the corresponding location relative to the reference location. The reference location is any location in the image that may be detected in the investigated images with a high reproducibility and likelihood. In act 84, a sequence alignment may be carried out for the different data sequences resulting in aligned sequences. Referring to FIG. 7, this may provide that sequence 75 is aligned to sequence 70. The alignment was also discussed above in connection with acts 350 and 360 in more detail. In act 85, an aggregated representation of the vessel is determined based on the aligned data sequences, which provides that features pertaining to the same location of the vessel are aggregated, and this aggregated representation provides a precise model that may then be used in act 86 for the determination of a flow parameter through the vessel. The flow parameter of the vessel represents the flow of blood through the vessel. The flow parameter may include parameters such as the fractional flow reserve (FFR), an index of microcirculatory resistance (IMR), and microvascular resistance reserve (MRR).

From the above, some general conclusions may be drawn. In this method, it is possible to determine in addition in each image of the plurality of images at least one additional feature of the first vessel and its distance to the reference location, where the sequence alignment may be carried out taking into account the distance of this at least one additional feature to the reference location. As discussed above, other features such as curvature or bifurcation points may be used at a sequence alignment to improve the alignment. This additional feature may include a bifurcation of the vessel, a vessel segment label, a stenosis marker, or any other feature that may be easily identified in the image.

The parameter that is determined in the different points in the vessel may represent a cross-sectional area available for the blood flow through the vessel. The parameter may include the diameter or the radius of the vessel at the corresponding location. This parameter may then be used to determine the actual flow surface for the blood through the vessel at the corresponding location.

The determination of the aggregated representation may include the aggregation of locations of the vessel belonging to the same location in the vessel. Here, it is possible to use the geometric mean for the parameters such as the radius or diameter.

For the aggregation of the distances belonging to the same location, it is possible to aggregate the distances to a reference location including aggregating a maximum difference between the current location and the previously aggregated location. This maximum distance is then used as new aggregated distance.

Further, it is possible that in each of the images, a curvature of the first vessel along the different points is determined, where determining the aggregated representation includes aggregating the curvature obtained at each of the different locations in the images.

The determined flow parameter may include the fractional flow reserve (FFR) or any other parameter such as index of microcirculatory resistance (IMR) or microvascular resistance reserve (MRR).

In one embodiment, the images are X-ray images. The images may include angiographic X-ray images of the heart, and the sequence alignment algorithm may include a Dynamic Time Warping algorithm or an Optimal Subsequence Bijection algorithm.

A comprehensive angio-based coronary assessment pipeline includes subtasks such as vessel detection, segmentation, branch labelling, fractional flow reserve (FFR) computation, etc. Aggregating information from multiple views is necessary because it provides a complete picture of the coronary anatomy, especially in the presence of eccentric stenosis. Single-view analysis often misses or misinterprets these irregularities, leading to inaccurate assessments. Therefore, multiple views enable better diagnostic accuracy. However, aggregating information extracted from multiple views poses major challenges.

Angio-based coronary assessment pipelines developed up to now (e.g., 3D QCA, FFR) may be employing a 3D reconstruction step having a goal that is to reconstruct the 3D vessel geometry based on the 2D geometries of the target vessel extracted in each view, and the geometric parameters of the acquisitions (e.g., primarily the projection matrix of the angiography system). This is achieved by performing a back-projection of the 2D geometries into the 3D space. However, this approach poses multiple challenges and requires preconditions, such as: Imposing a minimum angulation separation between views (e.g., >=30 degrees). Having a complete coverage of the target vessel in all the used views, which poses difficulties in certain angulations such as RCA RAO (Right coronary artery right anterior oblique) views and LCA LAO-CAU (Left Coronary artery, Left Anterior Oblique, with CAUDAL angulation) views due to foreshortening. Handling processing views that are not acquired simultaneously (e.g., using a biplane system), due to cardiac and respiratory movement, misalignment of cardiac phases, patient movement, table movement (e.g., panning between views), etc. Handling difficult reconstructing regions with severe vessel tortuosity due to apparent foreshortening in at least one of the projections. Ensuring accurate calibration of the angiography system to maintain the precision of the projection matrices.

Some of these challenges may be tackled by introducing an optimization procedure with regularization in the 3D reconstruction step and employing additional constraining information such as the location of the catheter tip along with locations of other fixed landmarks (e.g., stenoses and bifurcations, potentially provided via user input). However, the 3D reconstruction task may remain an ill-posed problem, and thus, a robust solution remains a big challenge.

The solution of the present embodiments discussed above circumvents the disadvantages of the full 3D reconstruction by allowing the result to be at 2.5D geometry so that the preconditions imposed by the 3D reconstructions are relaxed. Despite the fact that a full 3D geometry process with a full 3D mesh of the target vessel is not provided, the obtained information as discussed above is often sufficient for a coronary assessment such as the QCA and FFR computation. As a consequence, the approach of the present embodiments has the advantage of being able to process views independent of angulation, views that are not acquired simultaneously, and is also robust to movement such as cardiac and respiratory movement, patient movement, or table movement, and also enables vessel stitching.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method that is computer-implemented, the method comprising:
receiving a plurality of images of vessels, the plurality of images being obtained from different viewing angles relative to the vessels;
determining, in each image of the plurality of images and for a first vessel of the vessels, a distance of different points along the first vessel relative to a reference location at the first vessel;
determining, in each image of the plurality of images, and at each of the different points, a parameter of the first vessel for blood flow through the first vessel;
providing, for each image of the plurality of images, a data sequence indicating the parameter relative to a distance to the reference location;
carrying out a sequence alignment of the data sequences obtained for the plurality of images providing aligned data sequences;
determining an aggregated representation of the first vessel based on the aligned data sequences; and
determining a flow parameter for the first vessel from the aggregated representation.

2. The method of claim 1, further comprising determining, in each image of the plurality of images, for the first vessel at least one additional feature of the first vessel and a distance of the at least one additional feature to the reference location,
wherein the sequence alignment is carried out taking into account the distance of the at least one additional feature to the reference location.

3. The method of claim 2, wherein the at least one additional feature comprises a bifurcation of the first vessel, a vessel segment label, a stenosis marker, or any combination thereof.

4. The method of claim 1, wherein the parameter comprises a cross-sectional area available for the blood flow through the first vessel.

5. The method of claim 1, wherein the parameter comprises a cross-sectional area, a diameter, or a radius of the first vessel.

6. The method of claim 1, wherein determining the aggregated representation comprises aggregating features of the first vessel belonging to a same location in the first vessel.

7. The method of claim 1, wherein determining the aggregated representation comprises aggregating distances belonging to a same location,
wherein the distances to the reference location are aggregated with aggregating a maximum difference between a current location and a previously aggregated location.

8. The method of claim 1, further determining, for each image of the plurality of images, a curvature of the first vessel along the different points,
wherein determining the aggregated representation comprises aggregating the curvature at each of the different points.

9. The method of claim 1, wherein determining the flow parameter comprises determining a fractional flow reserve (FFR), an index of microcirculatory resistance (IMR), and a microvascular resistance reserve (MRR).

10. The method of claim 1, wherein the images are two-dimensional X-ray images.

11. The method of claim 1, wherein the sequence alignment a Dynamic Time Warping or an Optimal Subsequence Bijection.

12. A computing device comprising:
a processing unit; and
a memory comprising program code, wherein the processing unit, when executing the program code, is configured to:
receive a plurality of images of vessels, the plurality of images being obtained from different viewing angles relative to the vessels;
determine, in each image of the plurality of images and for a first vessel of the vessels, a distance of different points along the first vessel relative to a reference location at the first vessel;
determine, in each image of the plurality of images, and at each of the different points, a parameter of the first vessel for blood flow through the first vessel;
provide, for each image of the plurality of images, a data sequence indicating the parameter relative to a distance to the reference location;
carry out a sequence alignment of the data sequences obtained for the plurality of images providing aligned data sequences;
determine an aggregated representation of the first vessel based on the aligned data sequences; and
determine a flow parameter for the first vessel from the aggregated representation.

13. An angiography device comprising:
a computing device comprising:
a processing unit; and
a memory comprising program code, wherein the processing unit, when executing the program code, is configured to:
receive a plurality of images of vessels, the plurality of images being obtained from different viewing angles relative to the vessels;
determine, in each image of the plurality of images and for a first vessel of the vessels, a distance of different points along the first vessel relative to a reference location at the first vessel;
determine, in each image of the plurality of images, and at each of the different points, a parameter of the first vessel for blood flow through the first vessel;
provide, for each image of the plurality of images, a data sequence indicating the parameter relative to a distance to the reference location;
carry out a sequence alignment of the data sequences obtained for the plurality of images providing aligned data sequences;
determine an aggregated representation of the first vessel based on the aligned data sequences; and
determine a flow parameter for the first vessel from the aggregated representation.

14. In a non-transitory computer-readable storage medium that stores instructions executable by one or more processors, the instructions comprising:
receiving a plurality of images of vessels, the plurality of images being obtained from different viewing angles relative to the vessels;
determining, in each image of the plurality of images and for a first vessel of the vessels, a distance of different points along the first vessel relative to a reference location at the first vessel;
determining, in each image of the plurality of images, and at each of the different points, a parameter of the first vessel for blood flow through the first vessel;
providing, for each image of the plurality of images, a data sequence indicating the parameter relative to a distance to the reference location;
carrying out a sequence alignment of the data sequences obtained for the plurality of images providing aligned data sequences;
determining an aggregated representation of the first vessel based on the aligned data sequences; and
determining a flow parameter for the first vessel from the aggregated representation.

15. The non-transitory computer-readable storage medium of claim 14, wherein the instructions further comprise determining, in each image of the plurality of images, for the first vessel at least one additional feature of the first vessel and a distance of the at least one additional feature to the reference location,
wherein the sequence alignment is carried out taking into account the distance of the at least one additional feature to the reference location.

16. The non-transitory computer-readable storage medium of claim 15, wherein the at least one additional feature comprises a bifurcation of the first vessel, a vessel segment label, a stenosis marker, or any combination thereof.

17. The non-transitory computer-readable storage medium of claim 15, wherein the parameter comprises a cross-sectional area available for the blood flow through the first vessel.

18. The non-transitory computer-readable storage medium of claim 14, wherein the parameter comprises a cross-sectional area, a diameter, or a radius of the first vessel.
